(19) United States
(12) Patent Application Publication
(10) Pub. No.: US 2020/0165358 A1
(43) Pub. Date: May 28, 2020

(54) METHODS AND COMPOSITIONS FOR EVALUATION AND TREATMENT OF SYNUCLEINOPATHIES

(71) Applicant: Parkinson?s Institute, Mountain View, CA (US)

(72) Inventors: Carrolee BARLOW, Mountain View, CA (US); Tyler MOLITOR, Mountain View, CA (US); Huiying ZHANG, Mountain View, CA (US)

(21) Appl. No.: 16/692,382

(22) Filed: Nov. 22, 2019

Related U.S. Application Data
(60) Provisional application No. 62/772,063, filed on Nov. 27, 2018.

(51) Int. Cl. C07K 16/44; G01N 33/50; A61P 25/16; A61P 1/00

(52) U.S. Cl. CPC C07K 16/44; G01N 33/5008; A61K 38/00; A61P 1/00; A61P 25/16

(57) ABSTRACT

Provided herein are methods and compositions for diagnosis, treatment, and evaluation of one or more synucleinopathies in a subject. Methods may comprise detection of α-synuclein phosphorylation in intestinal cells of a subject. Methods may comprise inhibition of α-synuclein phosphorylation in intestinal cells of a subject.

### CROSS-REFERENCE

This application claims the benefit of U.S. Provisional Application No. 62/772,063 filed Nov. 27, 2018, which application is incorporated herein by reference.

### BACKGROUND

The accumulation and aggregation of α-synuclein protein in neuronal tissue is a critical event in the pathophysiology of synucleinopathies such as pure autonomic failure, multiple system atrophy, and Parkinson's disease. However, early detection and prevention of this process remains a challenge, as does evaluation of its treatment and prevention. Recognized herein is a need for improved methods and compositions for treatment, diagnosis, and evaluation of synucleinopathies, including Parkinson's disease.

### BRIEF SUMMARY

Provided herein, in some embodiments, is a method for evaluating a treatment for a synucleinopathy comprising: (a) providing the treatment to a subject having the synucleinopathy; (b) obtaining a sample comprising intestinal cells from the subject; and (c) measuring α-synuclein phosphorylation levels in the intestinal cells to evaluate the efficacy of the treatment. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, (c) comprises comparing the phosphorylation levels to a reference value, thereby evaluating the efficacy of the treatment. In some embodiments, the reference value is obtained by measuring α-synuclein phosphorylation levels in one or more subjects who have not received the treatment. In some embodiments, the method further comprises providing the treatment to the subject if the phosphorylation levels are reduced relative to the reference value. In some embodiments, the intestinal cells are enteroendocrine cells. In some embodiments, the method further comprises making a clinical decision based on the results of the measuring. In some embodiments, the synucleinopathy is Parkinson's disease, dementia with Lewy bodies, pure autonomic failure, or multiple system atrophy.

Provided herein, in some embodiments, is a method for evaluating a treatment for a synucleinopathy comprising: (a) obtaining a first sample comprising intestinal cells from a subject having the synucleinopathy; (b) measuring α-synuclein phosphorylation levels in the intestinal cells of the first sample; (c) providing the treatment to the subject; (d) obtaining a second sample comprising intestinal cells from the subject; (e) measuring α-synuclein phosphorylation levels in the intestinal cells of the second sample; and (f) comparing the phosphorylation levels in (e) to the phosphorylation levels in (b), thereby evaluating the treatment. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the method further comprises providing the treatment to the subject if the phosphorylation levels in (e) are reduced relative to the phosphorylation levels in (b). In some embodiments, the intestinal cells are enteroendocrine cells. In some embodiments, the synucleinopathy is Parkinson's disease, dementia with Lewy bodies, pure autonomic failure, or multiple system atrophy.

Provided herein, in some embodiments, is a method for clinical evaluation of a treatment for a synucleinopathy comprising: (a) providing the treatment to a subject having the synucleinopathy; (b) obtaining a sample comprising intestinal cells from the subject; (c) measuring α-synuclein phosphorylation levels in the intestinal cells; and (d) determining a clinical outcome based on the measuring. In some embodiments, determining the clinical outcome comprises determining an efficacy of the treatment in treating the synucleinopathy in the subject. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, (c) comprises comparing the phosphorylation levels to a reference value, wherein the clinical outcome is determined based on the comparing. In some embodiments, the synucleinopathy is Parkinson's disease, dementia with Lewy bodies, pure autonomic failure, or multiple system atrophy.

Provided herein, in some embodiments, is a method of determining a response to a treatment for a synucleinopathy in a subject in need thereof, comprising: (a) providing the treatment to the subject; (b) measuring a level of α-synuclein phosphorylation in a sample comprising intestinal cells obtained from the subject; and (c) comparing the level of α-synuclein phosphorylation in the sample to a reference value, thereby determining the response to the treatment. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, (c) comprises comparing the phosphorylation levels to a reference value, thereby evaluating the efficacy of the treatment. In some embodiments, the reference value is obtained by measuring α-synuclein phosphorylation levels in one or more subjects who have not received the treatment. In some embodiments, the intestinal cells are enteroendocrine cells. In some embodiments, the synucleinopathy is Parkinson's disease, dementia with Lewy bodies, pure autonomic failure, or multiple system atrophy.

Provided herein, in some embodiments, is a method of adjusting a treatment for a synucleinopathy in a subject in need thereof, comprising: (a) providing the treatment to the subject; (b) measuring a level of α-synuclein phosphorylation in a sample comprising intestinal cells obtained from the subject; (c) comparing the level of α-synuclein phosphorylation in the sample to a reference value; and (d) adjusting a treatment for the synucleinopathy based on the results of (c). In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, (c) comprises comparing the phosphorylation levels to a reference value, thereby evaluating the efficacy of the treatment. In some embodiments, the reference value is obtained by measuring α-synuclein phosphorylation levels in one or more subjects who have not received the treatment. In some embodiments, the intestinal cells are enteroendocrine cells. In some embodiments, the synucleinopathy is Parkinson's disease, dementia with Lewy bodies, pure autonomic failure, or multiple system atrophy.

Provided herein, in some embodiments, is a method of treating a synucleinopathy comprising modulating α-synuclein phosphorylation in intestinal cells. In some embodiments, modulating the α-synuclein phosphorylation comprises reducing the α-synuclein phosphorylation. In some embodiments, modulating the α-synuclein phosphorylation comprises using a compound capable of inhibiting α-synuclein phosphorylation. In some embodiments, the compound is a tyrosine kinase inhibitor. In some embodiments, the tyrosine kinase inhibitor is a c-abl kinase inhibitor. In some embodiments, the α-synuclein phosphorylation is phosphorylation on a tyrosine residue of α-synuclein. In some embodiments, the tyrosine residue is Y39. In some embodiments, modulating the α-synuclein phosphorylation does not comprise modulating phosphorylation on a serine residue. In some embodiments, the serine residue is S129. In some embodiments, the intestinal cells are enteroendocrine cells. In some embodiments, the synucleinopathy is Parkinson's disease, dementia with Lewy bodies, pure autonomic failure, or multiple system atrophy.

Provided herein, in some embodiments, is a method for treating a subject with a synucleinopathy comprising providing a compound capable of inhibiting α-synuclein phosphorylation to intestinal cells of the subject. In some embodiments, the compound is a tyrosine kinase inhibitor. In some embodiments, the tyrosine kinase inhibitor is a c-abl kinase inhibitor. In some embodiments, the method further comprises obtaining a sample comprising intestinal cells from the subject and measuring α-synuclein phosphorylation levels in the intestinal cells to evaluate the efficacy of the compound. In some embodiments, the intestinal cells are enteroendocrine cells. In some embodiments, the compound is not provided to neuronal cells. In some embodiments, the compound is unable to access a nerve cell of the subject. In some embodiments, the compound is formulated such that it is unable to access a nerve cell of the subject. In some embodiments, the compound inhibits α-synuclein phosphorylation on a tyrosine residue. In some embodiments, the tyrosine residue is Y39. In some embodiments, the compound does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of preventing the development of a synucleinopathy in a subject comprising providing a compound capable of inhibiting α-synuclein phosphorylation to intestinal cells of the subject. In some embodiments, the subject is at risk for the synucleinopathy. In some embodiments, the subject harbors one or more mutations associated with the synucleinopathy. In some embodiments, the one or more mutations are present in a gene selected from the group consisting of ADCY5, DNAJC13, MCOLN1, PRKRA, TDP43, ANO3, FBXO7, PANK2, PSEN1, THAP1, APOE, GBA, PARK2, PSEN2, TOR1A, APP, GCH1, PARK7, RAB12, VAC14, ATP13A2, GNAL, PDE8B, RAB39B, VPS13C, ATP9A, GNE, PDGFB, SGCE, VPS35, C19orf12, GRN, PDGFRB, SLC20A2, XDP, CHCHD2, KMT2B, PINK1, SNCA, XPR1, COX20, LRRK2, PLA2G6, SNCB, DCTN1, MAPT, POLG, TAF1, DNAJC13, ATP9A, VPS13C, DJ1, GBA, LRRK2, PARK2, PINK1, SNCA, VPS35, FBXO7, CHCHD2, Rab39B, TOR1A, SGCE, PRKRA, ANO3, GNAL, KMT2B, XDP, GCH1, THAP1, TAF1, ADCY5, Rab 12, SLC20A2, PDGFRB, PDGFB, XPR1, PANK2, C19orf12, PLA2G6, APP, APOE, PSEN1, PSEN2, GRN, TDP43, SNCB, DCTN1, MAPT, ATP13A2, COX20, POLG, MCOLN1, GNE, PDE8B, and VAC14. In some embodiments, the compound is a tyrosine kinase inhibitor. In some embodiments, the tyrosine kinase inhibitor is a c-abl kinase inhibitor. In some embodiments, the method further comprises obtaining a sample comprising intestinal cells from the subject and measuring α-synuclein phosphorylation levels in the intestinal cells to evaluate the efficacy of the compound. In some embodiments, the intestinal cells are enteroendocrine cells. In some embodiments, the compound is not provided to neuronal cells. In some embodiments, the compound is unable to access a nerve cell of the subject. In some embodiments, the compound is formulated such that it is unable to access a nerve cell of the subject. In some embodiments, the compound inhibits α-synuclein phosphorylation on a tyrosine residue. In some embodiments, the tyrosine residue is Y39. In some embodiments, the compound does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a subject with a synucleinopathy comprising: (a) providing a treatment to the subject; (b) obtaining a sample comprising intestinal cells from the subject; (c) measuring α-synuclein phosphorylation levels in the intestinal cells of the sample; (d) comparing the α-synuclein phosphorylation levels in (c) to a reference value. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the reference value is obtained by measuring α-synuclein phosphorylation levels in one or more subjects who have not received the treatment. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a subject with a synucleinopathy comprising: (a) obtaining a first sample comprising intestinal cells from a subject; (b) measuring α-synuclein phosphorylation levels in the intestinal cells of the first sample; (c) providing a treatment to the subject; (d) obtaining a second sample comprising intestinal cells from the subject; (e) measuring α-synuclein phosphorylation levels in the intestinal cells of the second sample; and (f) comparing the α-synuclein phosphorylation levels in the intestinal cells of the second sample with the α-synuclein phosphorylation levels in the intestinal cells of the second sample. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a subject with a synucleinopathy comprising: (a) providing a treatment to the subject; (b) obtaining a sample comprising intestinal cells from the subject; (c) measuring α-synuclein phosphorylation levels in the intestinal cells of the sample; (d) comparing the α-synuclein phosphorylation levels in (c) to a reference value; (e) if the α-synuclein levels in (c) are significantly greater than or equal to the reference value, providing the treatment at a higher concentration relative to (a); and (f) if the α-synuclein phosphorylation levels in (c) are significantly less than the reference value, providing the treatment at a lower or equal concentration relative to (a). In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the reference value is obtained by measuring α-synuclein phosphorylation levels in one or more subjects who have not received the treatment. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a subject with a synucleinopathy comprising: (a) obtaining a first sample comprising intestinal cells from a subject; (b) measuring α-synuclein phosphorylation levels in the intestinal cells of the first sample; (c) providing a treatment to the subject; (d) obtaining a second sample comprising intestinal cells from the subject; (e) measuring α-synuclein phosphorylation levels in the intestinal cells of the second sample; (f) if the α-synuclein phosphorylation levels in the intestinal cells of the second sample are significantly greater than or equal to the α-synuclein phosphorylation levels in the first sample, providing the treatment at a higher concentration relative to (c); and (g) if the α-synuclein phosphorylation levels in the intestinal cells of the second sample are significantly less than the α-synuclein phosphorylation levels in the first sample, providing the treatment at a lower or equal concentration relative to (c). In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a synucleinopathy in a subject in need thereof, comprising: (a) screening the subject for an abnormal level of α-synuclein phosphorylation; (b) providing a treatment to the subject; (c) measuring a level of α-synuclein phosphorylation in a sample comprising intestinal cells obtained from the subject; (d) comparing the level of α-synuclein phosphorylation in the sample to a reference value; and (e) adjusting the treatment provided to the subject based on the difference in the level of α-synuclein phosphorylation between the sample and the reference value. In some embodiments, measuring the level of α-synuclein phosphorylation in the sample comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of treating a synucleinopathy in a subject in need thereof, comprising: (a) providing a treatment to the subject; (b) measuring a level of α-synuclein phosphorylation in a sample comprising intestinal cells obtained from the subject; (c) comparing the level of α-synuclein phosphorylation in the sample to a reference value; and (d) adjusting the treatment provided to the subject based on the difference in the level of α-synuclein phosphorylation between the sample and the reference value. In some embodiments, measuring the level of α-synuclein phosphorylation in the sample comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the treatment is a tyrosine kinase inhibitor. In some embodiments, the tyrosine kinase inhibitor is a c-abl inhibitor. In some embodiments, the treatment inhibits α-synuclein phosphorylation on a tyrosine residue. In some embodiments, the tyrosine residue is Y39. In some embodiments, the treatment does not inhibit α-synuclein phosphorylation on a serine residue. In some embodiments, the serine residue is S129. In some embodiments, the synucleinopathy is Parkinson's disease, dementia with Lewy bodies, pure autonomic failure, or multiple system atrophy.

Provided herein, in some embodiments, is a method of detecting α-synuclein phosphorylation in a subject comprising: (a) obtaining a sample comprising intestinal cells from the subject; and (b) detecting α-synuclein phosphorylation at a tyrosine residue in the intestinal cells by contacting the sample with an antibody that specifically binds to α-synuclein when the α-synuclein is phosphorylated at a tyrosine residue, wherein the antibody does not bind α-synuclein when not phosphorylated at the tyrosine residue. In some embodiments, the subject has or is suspected of having a synucleinopathy. In some embodiments, the tyrosine residue is Y39. In some embodiments, the intestinal cells are enteroendocrine cells. In some embodiments, the synucleinopathy is Parkinson's disease, dementia with Lewy bodies, pure autonomic failure, or multiple system atrophy.

Provided herein, in some embodiments, is a method for diagnosing a subject suspected of having a synucleinopathy comprising: (a) measuring α-synuclein phosphorylation levels in the intestinal cells of the subject; and (b) comparing the α-synuclein phosphorylation levels to a reference value, thereby diagnosing the subject with the synucleinopathy. In some embodiments, measuring the phosphorylation levels comprises using an antibody specific for phosphorylated α-synuclein. In some embodiments, the antibody is specific for α-synuclein when phosphorylated on a tyrosine residue. In some embodiments, the tyrosine residue is Y39. In some embodiments, the reference value is obtained by measuring α-synuclein phosphorylation levels in one or more subjects who do not have the synucleinopathy. In some embodiments, α-synuclein phosphorylation levels in the intestinal cells of the subject are greater than the reference value. In some embodiments, diagnosing the subject comprises identifying the subject as harboring one or more mutations associated with the synucleinopathy. In some embodiments, the one or more mutations are present in a gene selected from the group consisting of ADCY5, DNAJC13, MCOLN1, PRKRA, TDP43, ANO3, FBXO7, PANK2, PSEN1, THAP1, APOE, GBA, PARK2, PSEN2, TOR1A, APP, GCH1, PARK7, RAB12, VAC14, ATP13A2, GNAL, PDE8B, RAB39B, VPS13C, ATP9A, GNE, PDGFB, SGCE, VPS35, C19orf12, GRN, PDGFRB, SLC20A2, XDP, CHCHD2, KMT2B, PINK1, SNCA, XPR1, COX20, LRRK2, PLA2G6, SNCB, DCTN1, MAPT, POLG, TAF1, DNAJC13, ATP9A, VPS13C, DJ1, GBA, LRRK2, PARK2, PINK1, SNCA, VPS35, FBXO7, CHCHD2, Rab39B, TOR1A, SGCE, PRKRA, ANO3, GNAL, KMT2B, XDP, GCH1, THAP1, TAF1, ADCY5, Rab 12, SLC20A2, PDGFRB, PDGFB, XPR1, PANK2, C19orf12, PLA2G6, APP, APOE, PSEN1, PSEN2, GRN, TDP43, SNCB, DCTN1, MAPT, ATP13A2, COX20, POLG, MCOLN1, GNE, PDE8B, and VAC14. In some embodiments, the one or more mutations are present in SNCA. In some embodiments, the method further comprises providing a treatment recommendation for treating the synucleinopathy in the subject. In some embodiments, the method further comprises providing a treatment for the synucleinopathy to the subject. In some embodiments, the synucleinopathy is Parkinson's disease, dementia with Lewy bodies, pure autonomic failure, or multiple system atrophy.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in their entireties.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows the results from the experiments described in Example 2.

### DETAILED DESCRIPTION

### Overview

Synucleinopathies, or neurological disorders characterized by aggregate, mutant, or otherwise abnormal α-synuclein protein, are a serious health challenge. One strategy for treatment of these disorders comprises prevention of the spread of aggregate or abnormal α-synuclein protein from intestinal cells to the enteric nervous system (ENS), and then from the ENS into the central nervous system (CNS). Recognized herein is a need for methods of evaluating the development and progression of synucleinopathies, and for new methods for treating synucleinopathies by preventing the spread of aggregate or abnormal α-synuclein protein.

Disclosed herein are methods and compositions for diagnosing, treating, and evaluating the presence, development, and progression of synucleinopathies. Also disclosed are methods for detecting α-synuclein phosphorylation in intestinal cells of a subject. In some examples, detection of phosphorylation levels of α-synuclein on a tyrosine 39 (Y39) residue in intestinal cells (e.g., enteroendocrine cells) of a subject may be used to diagnose the subject with a synucleinopathy and/or evaluate treatment of a synucleinopathy.

In some aspects, inhibition of α-synuclein phosphorylation in intestinal cells may be used in the treatment of a synucleinopathy. In some examples, inhibition of α-synuclein phosphorylation on a tyrosine 39 (Y39) residue in intestinal cells (e.g., enteroendocrine cells) of a subject with a synucleinopathy may be used to treat the subject. This may be accomplished by providing a compound capable of inhibiting α-synuclein phosphorylation. In some cases, a compound capable of inhibiting α-synuclein phosphorylation may be a tyrosine kinase inhibitor, such that Y39 phosphorylation is inhibited.

### Definitions

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to ±10%. In some embodiments, the term “about” refers to ±5%.

The term “symptom” refers to a subjective evidence of a disease, such as altered gait, as perceived by the patient. A “sign” refers to objective evidence of a disease as observed by a physician.

“Inhibition,” “treatment,” and “treating” are used interchangeably and refer to, for example, stasis of symptoms, prolongation of survival, partial or full amelioration of symptoms, and partial or full eradication of a condition, disease, or disorder.

A “subject,” refers to a living organism such as mammals. Examples of subjects include, but are not limited to, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks) fish (e.g., sharks) or frogs (e.g., Xenopus), and non-mammalian invertebrates, as well as transgenic species thereof. In certain aspects, a subject refers to a single organism (e.g., human).

“Treat” or “treatment” refers to a therapeutic treatment wherein the object is to eliminate or lessen symptoms.

### Detecting α-Synuclein Phosphorylation in Intestinal Cells

As recognized herein, α-synuclein phosphorylation may be present in intestinal cells. Detection of α-synuclein phosphorylation on one or more residues in intestinal cells may be useful in, for example, diagnosis, treatment, or evaluation of a synucleinopathy. Detection of α-synuclein phosphorylation may comprise detection of phosphorylation on a tyrosine 39 (Y39) residue of the human α-synuclein protein, or its corresponding residue on an α-synuclein protein from other organisms. In one example, α-synuclein phosphorylation levels in intestinal cells of a subject may be used to diagnose a subject as having a synucleinopathy. In another example, α-synuclein phosphorylation levels in intestinal cells of a subject may be detected to evaluate the efficacy of a synucleinopathy treatment.

Disclosed herein are methods for detecting α-synuclein phosphorylation in intestinal cells of a subject. In some cases, a sample comprising intestinal cells may be obtained from a subject. Next, α-synuclein phosphorylation may be detected at a tyrosine residue in the intestinal cells by contacting the sample with an antibody that specifically binds to α-synuclein when the α-synuclein is phosphorylated at a tyrosine residue, where the antibody does not bind α-synuclein when not phosphorylated at the tyrosine residue. The subject may be suspected of having a synucleinopathy. The subject may have a synucleinopathy. The subject may not have a synucleinopathy (e.g., may be a healthy subject). The intestinal cells may comprise enteroendocrine cells. The tyrosine residue may be Y39 of a human α-synuclein protein, or the corresponding residue of an α-synuclein protein from another organism.

An antibody, or antigen binding fragment thereof, may be used to detect α-synuclein phosphorylation in intestinal cells of a subject. An antibody may bind specifically when α-synuclein is phosphorylated at one or more residues. An antibody may not bind when α-synuclein is not phosphorylated at the one or more residues. An antibody may bind specifically when α-synuclein is phosphorylated at one or more tyrosine residues. In some cases, an antibody binds specifically when α-synuclein is phosphorylated at Y39 of a human α-synuclein protein, or the corresponding residue of an α-synuclein protein from another organism.

### Synucleinopathy Diagnosis

Disclosed herein are methods for diagnosis of a synucleinopathy. In some cases, α-synuclein phosphorylation levels may be measured in intestinal cells of a subject. The α-synuclein phosphorylation levels may be tyrosine phosphorylation levels. In one example, measuring α-synuclein phosphorylation levels comprises measuring phosphorylation of α-synuclein at a tyrosine 39 (Y39) residue of a human α-synuclein protein. Next, α-synuclein phosphorylation levels may be compared to a reference value, thereby diagnosing the subject with the synucleinopathy.

For example, a subject may be suspected of having a synucleinopathy. Intestinal cells from the subject may be taken and α-synuclein phosphorylation levels measured. The intestinal cells may be enteroendocrine cells. These α-synuclein phosphorylation levels may be compared to a reference value. A reference value may be obtained by measuring α-synuclein phosphorylation levels from one or more subjects who do not have the synucleinopathy (e.g., healthy subjects). A subject may be diagnosed with a synucleinopathy by identifying an increase in α-synuclein phosphorylation levels relative to the reference value.

Diagnosing a subject with the synucleinopathy may comprise identifying the subject as harboring one or more mutations associated with the synucleinopathy. A subject with a synucleinopathy may have one or more mutations in one or more genes including, but not limited to, ADCY5, DNAJC13, MCOLN1, PRKRA, TDP43, ANO3, FBXO7, PANK2, PSEN1, THAP1, APOE, GBA, PARK2, PSEN2, TOR1A, APP, GCH1, PARK7, RAB12, VAC14, ATP13A2, GNAL, PDE8B, RAB39B, VPS13C, ATP9A, GNE, PDGFB, SGCE, VPS35, C19orf12, GRN, PDGFRB, SLC20A2, XDP, CHCHD2, KMT2B, PINK1, SNCA, XPR1, COX20, LRRK2, PLA2G6, SNCB, DCTN1, MAPT, POLG, TAF1, DNAJC13, ATP9A, VPS13C, DJ1, GBA, LRRK2, PARK2, PINK1, SNCA, VPS35, FBXO7, CHCHD2, Rab39B, TOR1A, SGCE, PRKRA, ANO3, GNAL, KMT2B, XDP, GCH1, THAP1, TAF1, ADCY5, Rab 12, SLC20A2, PDGFRB, PDGFB, XPR1, PANK2, C19orf12, PLA2G6, APP, APOE, PSEN1, PSEN2, GRN, TDP43, SNCB, DCTN1, MAPT, ATP13A2, COX20, POLG, MCOLN1, GNE, PDE8B, or VAC14. For example, identifying an increase in α-synuclein phosphorylation levels relative to a reference value may indicate the presence of a mutation in the SCNA gene.

A synucleinopathy may be Parkinson's disease, dementia with Lewy bodies, pure autonomic failure, or multiple system atrophy (MSA). In some cases, a synucleinopathy is Parkinson's disease. In some cases, a synucleinopathy is dementia with Lewy bodies. In some cases, a synucleinopathy is pure autonomic failure. In some cases, a synucleinopathy is MSA.

### Synucleinopathy Treatment

Modulation of α-synuclein phosphorylation in intestinal cells of a subject may be useful in treatment of a synucleinopathy. Disclosed herein are methods for treatment of a synucleinopathy comprising modulating α-synuclein phosphorylation in intestinal cells (e.g., enteroendocrine cells) of a subject. In some cases, modulating α-synuclein phosphorylation comprises reducing α-synuclein phosphorylation levels. Reducing α-synuclein phosphorylation levels may comprise the use of one or more compounds capable of inhibiting α-synuclein phosphorylation at one or more residues (e.g., tyrosine residues). A compound for treating a synucleinopathy may be a tyrosine kinase inhibitor. A tyrosine kinase inhibitor may be, for example, a c-abl inhibitor. A compound may be capable of inhibiting α-synuclein phosphorylation in intestinal cells at a tyrosine residue (e.g., Y39), and may not be capable of inhibiting α-synuclein phosphorylation at a serine residue (e.g., S129). For example, modulating α-synuclein phosphorylation levels may comprise reducing Y39 phosphorylation levels while not altering S129 phosphorylation levels.

In some cases, a treatment is provided to a subject having a synucleinopathy. A treatment may be capable of inhibiting α-synuclein phosphorylation levels in intestinal cells of the subject. Next, a sample comprising intestinal cells of the subject may be obtained. The sample may be, for example, a biopsy sample. Next, α-synuclein phosphorylation levels in the intestinal cells may be measured. For example, α-synuclein phosphorylation levels may be measured using an antibody, or antigen binding fragment thereof, which is specific for α-synuclein when phosphorylated at one or more residues (e.g., tyrosine residues). Finally, the measured α-synuclein levels may be compared to a reference value. A reference value may be obtained by measuring α-synuclein phosphorylation levels in intestinal cells of one or more subjects who do not have a synucleinopathy (e.g., healthy subjects). Comparing α-synuclein phosphorylation levels to a reference value may be useful in, for example, evaluating the efficacy of a treatment, adjusting a treatment dosage level, changing a type of treatment provided, etc. In some examples, if the α-synuclein phosphorylation levels measured in the subject are greater than or equal to the reference value, the treatment is provided to the subject at a higher concentration than initially provided, while, if the α-synuclein phosphorylation levels measured in the subject are less than the reference value, the treatment is provided to the subject at a lower concentration than initially provided.

A subject treated with the disclosed methods may have a synucleinopathy. A subject with a synucleinopathy may have one or more mutations in one or more genes including, but not limited to, ADCY5, DNAJC13, MCOLN1, PRKRA, TDP43, ANO3, FBXO7, PANK2, PSEN1, THAP1, APOE, GBA, PARK2, PSEN2, TOR1A, APP, GCH1, PARK7, RAB12, VAC14, ATP13A2, GNAL, PDE8B, RAB39B, VPS13C, ATP9A, GNE, PDGFB, SGCE, VPS35, C19orf12, GRN, PDGFRB, SLC20A2, XDP, CHCHD2, KMT2B, PINK1, SNCA, XPR1, COX20, LRRK2, PLA2G6, SNCB, DCTN1, MAPT, POLG, TAF1, DNAJC13, ATP9A, VPS13C, DJ1, GBA, LRRK2, PARK2, PINK1, SNCA, VPS35, FBXO7, CHCHD2, Rab39B, TOR1A, SGCE, PRKRA, ANO3, GNAL, KMT2B, XDP, GCH1, THAP1, TAF1, ADCY5, Rab 12, SLC20A2, PDGFRB, PDGFB, XPR1, PANK2, C19orf12, PLA2G6, APP, APOE, PSEN1, PSEN2, GRN, TDP43, SNCB, DCTN1, MAPT, ATP13A2, COX20, POLG, MCOLN1, GNE, PDE8B, or VAC14. A synucleinopathy may be Parkinson's disease, dementia with Lewy bodies, pure autonomic failure, or multiple system atrophy. In some cases, the disclosed methods comprise methods of treating a subject with Parkinson's disease.

### Evaluation of Synucleinopathy Treatment

As disclosed herein, detection of α-synuclein phosphorylation levels in intestinal cells of a subject may be useful in evaluating a treatment of a subject with a synucleinopathy. In some cases, a treatment may be provided to a subject with a synucleinopathy. Next, a sample may be obtained comprising intestinal cells of the subject. The sample may be, for example, a biopsy sample. Finally, α-synuclein phosphorylation levels may be measured in the intestinal cells to evaluate the efficacy of the treatment. Measuring the α-synuclein phosphorylation levels may comprise the use of an antibody specific for one or more residues (e.g., tyrosine residues) of an α-synuclein protein. In some cases, measuring α-synuclein phosphorylation levels comprises use of an antibody specific for a Y39 residue of α-synuclein.

Measured α-synuclein phosphorylation levels may be compared to a reference value, thereby determining the efficacy of a treatment. For example, α-synuclein phosphorylation levels may be compared to a reference value obtained from measuring α-synuclein phosphorylation levels in one or more subjects who do not have a synucleinopathy (e.g., healthy subjects). In this example, if the α-synuclein phosphorylation levels in the subject are equal to or less than the reference value, the treatment may be identified as effective.

Alternatively or in addition, α-synuclein phosphorylation levels may be measured both before and after a treatment. In some cases, a first sample comprising intestinal cells may be obtained from a subject, and α-synuclein phosphorylation levels may be measured in the intestinal cells of the first sample. Next, a treatment may be provided to a subject having a synucleinopathy. A second sample may then be obtained comprising intestinal cells from the subject, and α-synuclein phosphorylation levels may be measured in the intestinal cells of the second sample. The α-synuclein phosphorylation levels from the first sample and the second sample may be compared, thereby evaluating the efficacy of the treatment. For example, if the α-synuclein phosphorylation levels from the second (post-treatment) sample are reduced relative to the α-synuclein phosphorylation levels from the first (pre-treatment) sample, then the treatment may be identified as effective. Conversely, if the α-synuclein phosphorylation levels from the second (post-treatment) sample are greater than or equal to the α-synuclein phosphorylation levels from the first (pre-treatment) sample, then the treatment may be identified as ineffective. This may be useful in informing treatment decisions, such as dosage modification, cessation of treatment, modification of treatment type, etc. A clinical outcome may be determined based on the treatment evaluation methods. A treatment evaluation method may identify a treatment as effective, thereby identifying a positive clinical outcome. Conversely, a treatment evaluation method may identify a treatment as ineffective, thereby identifying a negative clinical outcome.

A synucleinopathy may be Parkinson's disease, dementia with Lewy bodies, pure autonomic failure, or multiple system atrophy (MSA). In some cases, a synucleinopathy is Parkinson's disease. In some cases, a synucleinopathy is dementia with Lewy bodies. In some cases, a synucleinopathy is pure autonomic failure. In some cases, a synucleinopathy is MSA.

### Dosage Optimization

Dosage of a therapeutic used in treating a synucleinopathy may be optimized using methods described herein. In some cases, a subject may be administered a first dosage level of a therapeutic agent for a number of doses at a dosing frequency. Then, a first measurement of α-synuclein phosphorylation levels may be obtained from intestinal tissue of the subject. Next, the subject may be administered an increased dosage level, relative to the first dosage level, of the therapeutic agent for a number of doses at a dosing frequency, followed by a second measurement of α-synuclein phosphorylation levels from intestinal tissue of the subject. The first measurement and the second measurement may be compared to each other and/or to one or more reference values. Next, an optimal dosage level may be selected from the two dosage levels administered, based on a change in the α-synuclein phosphorylation levels and initiation of, if any, side effects. A reduction in α-synuclein phosphorylation levels may indicate an increase in the effectiveness of a dosage level. Iterations of increasing dosage level and measurement of α-synuclein phosphorylation levels testing may be administered until a maximum dosage level is achieved, no further change in α-synuclein phosphorylation is detected, or side effects outweigh benefits of treatment.

A therapeutic agent may be capable of inhibiting phosphorylation of α-synuclein in intestinal cells of a subject. A therapeutic agent may be capable of inhibiting phosphorylation of α-synuclein at one or more tyrosine residues. A therapeutic agent may be capable of inhibiting phosphorylation of α-synuclein at tyrosine 39 (Y39). A therapeutic agent may be capable of inhibiting phosphorylation of α-synuclein at Y39 without inhibition of α-synuclein phosphorylation at one or more serine residues (e.g., S129). A therapeutic agent may be a kinase inhibitor. A kinase inhibitor may be a tyrosine kinase inhibitor. A tyrosine kinase inhibitor may be a c-abl inhibitor. A synucleinopathy may be Parkinson's disease, dementia with Lewy bodies, pure autonomic failure, or multiple system atrophy (MSA). In some cases, a synucleinopathy is Parkinson's disease. In some cases, a synucleinopathy is dementia with Lewy bodies. In some cases, a synucleinopathy is pure autonomic failure. In some cases, a synucleinopathy is MSA.

A subject may be administered a first dosage level of a therapeutic agent which is below the recommended dosage. A subject may be administered an initial dosage which is the maximum recommended dose. A dosage level may be about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, or about 50 mg. The number of doses may be 1, 2, 3, 4, 5, 6, 7, 8, 9, 10, 12, 14, 16, 18, 20, 25, 30, 35, 40, 50, or 60 doses. The dosing frequency may be one hour, 2 hours, 3 hours, 4 hours, 5 hours, 6 hours, 7 hours, 8 hours, 9 hours, 10 hours, 11 hours, 12 hours, 14 hours, 16 hours, 18 hours, 20 hours, 22 hours, one day, 2 days, 3 days, 4 days, 5 days 6 days, one week, 2 weeks, 3 weeks, 4 weeks, one month, 2 months, 3 months, 4 months, 5 months, 6 months, or one year. Dosage level may be increased by about 5%, about 10%, about 20%, about 30%, about 40%, about 50%, about 60%, about 70%, about 80%, about 90%, or about 100%. Dosage level may be decreased by about 5%, about 10%, about 20%, about 30%, about 40%, about 50%, about 60%, about 70%, about 80%, about 90%, or about 100%.

### Neurological Disorders

“Neurological disorders,” as used herein, include Parkinson's disease, Multisystem Lewy body disease (MLBD), parkinsonism, dementia with Lewy bodies (DLB), pure autonomic failure (PAF), Parkinson's disease dementia (PDD), multiple system atrophy, progressive supranuclear palsy, corticobasal degeneration, fronto-temporal dementia, Alzheimer's disease without Parkinson's disease, atypical parkinsonism, α-synuclein- or tau-related neuropathy, Lewy neurites or neuronal cell loss in substantia nigra, and α-synuclein-positive Lewy bodies. A neurological disorder may be a synucleinopathy.

“Synucleinopathies,” as used herein, include dementia with Lewy bodies (DLB), pure autonomic failure (PAF), multiple system atrophy, and Parkinson's disease. In some cases, a synucleinopathy is dementia with Lewy bodies. In some cases, a synucleinopathy is pure autonomic failure. In some cases, a synucleinopathy is multiple system atrophy. In some cases, a synucleinopathy is Parkinson's disease. A subject with a synucleinopathy may have one or more mutations in one or more genes including, but not limited to, ADCY5, DNAJC13, MCOLN1, PRKRA, TDP43, ANO3, FBXO7, PANK2, PSEN1, THAP1, APOE, GBA, PARK2, PSEN2, TOR1A, APP, GCH1, PARK7, RAB12, VAC14, ATP13A2, GNAL, PDE8B, RAB39B, VPS13C, ATP9A, GNE, PDGFB, SGCE, VPS35, C19orf12, GRN, PDGFRB, SLC20A2, XDP, CHCHD2, KMT2B, PINK1, SNCA, XPR1, COX20, LRRK2, PLA2G6, SNCB, DCTN1, MAPT, POLG, TAF1, DNAJC13, ATP9A, VPS13C, DJ1, GBA, LRRK2, PARK2, PINK1, SNCA, VPS35, FBXO7, CHCHD2, Rab39B, TOR1A, SGCE, PRKRA, ANO3, GNAL, KMT2B, XDP, GCH1, THAP1, TAF1, ADCY5, Rab 12, SLC20A2, PDGFRB, PDGFB, XPR1, PANK2, C19orf12, PLA2G6, APP, APOE, PSEN1, PSEN2, GRN, TDP43, SNCB, DCTN1, MAPT, ATP13A2, COX20, POLG, MCOLN1, GNE, PDE8B, or VAC14.

### Subject

The methods disclosed herein may be used to evaluate a subject. A subject may include for example, a male or female adult, child, newborn, or fetus. A subject may be any target of therapeutic administration. A subject may be a test subject or a reference subject. A subject may be associated with a condition or disease or disorder, asymptomatic or symptomatic, have increased or decreased susceptibility to a disease or disorder, be associated or unassociated with a treatment or treatment regimen, or any combination thereof. As used in the present disclosure, a cohort may represent an ethnic group, a patient group, a particular age group, a group not associated with a particular disease or disorder, a group associated with a particular disease or disorder, a group of asymptomatic subjects, a group of symptomatic subjects, or a group or subgroup of subjects associated with a particular response to a treatment regimen or clinical trial. A subject may be suffering from a disorder, for example, a neurological disorder. A subject may be a test subject, a patient, or a candidate being treated with one or more therapeutics, wherein the subject, patient, or candidate is evaluated for treatment efficacy by one or more methods of the present disclosure. A subject may be evaluated by, for example, measuring α-synuclein phosphorylation levels, as disclosed elsewhere herein.

Subjects of all ages are contemplated in the present disclosure. Subjects may be from specific age subgroups, such as those over the age of 1, over the age of 2, over the age of 3, over the age of 4, over the age of 5, over the age of 6, over the age of 7, over the age of 8, over the age of 9, over the age of 10, over the age of 15, over the age of 20, over the age of 25, over the age of 30, over the age of 35, over the age of 40, over the age of 45, over the age of 50, over the age of 55, over the age of 60, over the age of 65, over the age of 70, over the age of 75, over the age of 80, or over the age of 85. Other embodiments of the disclosure pertain to other age groups, such as subjects aged less than age 85, less than age 80, less than age 75, less than age 70, less than age 65, less than age 60, less than age 55, less than age 50, less than age 45, less than age 40, less than age 35, less than age 30, less than age 25, less than age 20, less than age 15, less than age 10, less than age 9, less than age 8, less than age 6, less than age 5, less than age 4, less than age 3, less than age 2, or less than age 1. Other embodiments relate to subjects with age at onset of the disease in any of particular age or age ranges defined by the numerical values described in the above or other numerical values bridging these numbers. It is also contemplated that a range of ages may be relevant in certain embodiments, such as age at onset at more than age 15 but less than age 20. Other age ranges are however also contemplated, including all age ranges bracketed by the age values listed in the above.

### EXAMPLES

### Example 1

Generation of an α-synuclein-dependent mouse model of enteric nervous system neurodegeneration

An α-synuclein-dependent model of gastrointestinal (GI) dysfunction in mice was developed. This model displayed a markedly delayed whole gut transit time (WGTT) in animals harboring a pathogenic version of α-synuclein. The transgenic mouse models were developed on a FVB;129S6 Snca^{-/-} background harboring either two copies (2n) of the wild type human transgenic α-synuclein locus (PAC-Tg SNCA WT) or four copies (4n) of the aggregate prone A53T allele (dbl-PAC-Tg SNCA A53T). These mice were subjected to mixed gender mice assessment of their whole gut transit time by administering a non-absorbable Carmine dye by oral gavage and recording the time it takes for the red dye to be observed in the stool. Key findings from the behavioral assessment demonstrated that the dbl-PAC-Tg SNCA A53T strain displayed a significant functional GI deficit and progressive worsening of the deficit. Both of these phenotypes are observed in Parkinson's disease patients.

### Example 2

Assessment of pathogenic α-synuclein in murine GI tract

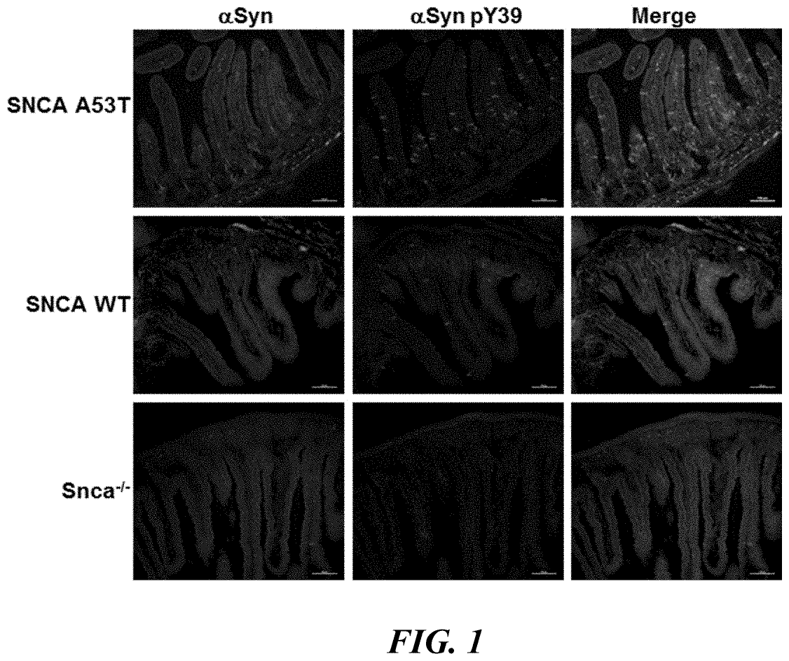

Immunohistological techniques were used to identify pathological phosphorylated α-synuclein species in the GI tract of the transgenic animals described in Example 1. GI tissues were probed with antibodies raised against total α-synuclein, phospho-Ser129 (pS129) α-synuclein and phospho-tyr39 (pY39) α-synuclein. Notably, when GI tissue from the SNCA A53T animals were probed with a phospho-specific antibody generated to specifically recognize phospho-tyrosine 39 on α-synuclein, there was a robust staining of cells outside the ENS (FIG. 1). These cells were enteroendocrine cells (EECs). The pY39 positive cells were separate from the pS129 α-synuclein, and were not present in ENS tissue. These results demonstrated specific pY39 staining of resident EECs in the GI tract.

FIG. 1 shows the results from the immunohistological staining of the intestinal cells from the animals. The left panels show a total α-synuclein antibody labeling resident neurons of the ENS in the myenteric plexus, submucosal plexus and lamina propria of the villus in SNCA A53T (top left panel) and SNCA WT (middle left panel) but not Snca^{-/-} tissues (bottom left panel). The middle panels show labeling using an antibody specific for pY39 α-synuclein. The staining of SNCA A53T tissue (top center panel) shows highly specific labeling of EECs. The incidence of the signal is significantly reduced in SNCA WT tissue (center panel) and is absent in Snca^{-/-} tissue (bottom center panel). Scale bars=100 μm.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for evaluating a treatment for a synucleinopathy comprising:
(a) providing the treatment to a subject having the synucleinopathy;
(b) obtaining a sample comprising intestinal cells from the subject; and
(c) measuring α-synuclein phosphorylation levels in the intestinal cells to evaluate the efficacy of the treatment.

2. The method of claim 1, wherein measuring the phosphorylation levels comprises using an antibody specific for phosphorylated α-synuclein.

3. The method of claim 2, wherein the antibody is specific for α-synuclein when phosphorylated on a tyrosine residue.

4. The method of claim 3, wherein the tyrosine residue is Y39.

5. The method of claim 1, wherein the treatment is a tyrosine kinase inhibitor.

6. (canceled)

7. The method of claim 1, wherein (c) comprises comparing the phosphorylation levels to a reference value, thereby evaluating the efficacy of the treatment.

8. (canceled)

9. (canceled)

10. The method of claim 1, wherein the intestinal cells are enteroendocrine cells.

11. (canceled)

12. The method of claim 1, wherein the synucleinopathy is Parkinson's disease, dementia with Lewy bodies, pure autonomic failure, or multiple system atrophy.

13.-61. (canceled)

62. A method for treating a subject with a synucleinopathy comprising providing a compound capable of inhibiting α-synuclein phosphorylation to intestinal cells of the subject; wherein the compound is a tyrosine kinase inhibitor.

63. (canceled)

64. (canceled)

65. The method of claim 62, further comprising obtaining a sample comprising intestinal cells from the subject and measuring α-synuclein phosphorylation levels in the intestinal cells to evaluate the efficacy of the compound.

66. The method of claim 62, wherein the intestinal cells are enteroendocrine cells.

67. The method of claim 62, wherein the compound is not provided to neuronal cells.

68. (canceled)

69. (canceled)

70. The method of claim 62, wherein the compound inhibits α-synuclein phosphorylation on a tyrosine residue.

71. The method of claim 70, wherein the tyrosine residue is Y39.

72. (canceled)

73. (canceled)

74. The method of claim 62, wherein the synucleinopathy is Parkinson's disease, dementia with Lewy bodies, pure autonomic failure, or multiple system atrophy.

75.-158. (canceled)

159. A method of detecting α-synuclein phosphorylation in a subject comprising:
(a) obtaining a sample comprising intestinal cells from the subject; and
(b) detecting α-synuclein phosphorylation at a tyrosine residue in the intestinal cells by contacting the sample with an antibody that specifically binds to α-synuclein when the α-synuclein is phosphorylated at a tyrosine residue, wherein the antibody does not bind α-synuclein when not phosphorylated at the tyrosine residue.

160. The method of claim 159, wherein the subject has or is suspected of having a synucleinopathy.

161. The method of claim 159, wherein the tyrosine residue is Y39.

162. The method of claim 159, wherein the intestinal cells are enteroendocrine cells.

163. The method of claim 160, wherein the synucleinopathy is Parkinson's disease, dementia with Lewy bodies, pure autonomic failure, or multiple system atrophy.

164.-175. (canceled)
